(19) United States
(12) Patent Application Publication
(10) Pub. No.: US 2021/0228622 A1
(43) Pub. Date: Jul. 29, 2021

(54) COMPOSITIONS OF OXYHYDROGEN AND THE THEREAPEUTIC USE THEREOF FOR OCULAR CONDITIONS

(71) Applicant: KHJ Biological LLC, Phoenix, AZ (US)

(72) Inventors: Kris Kesler, Phoenix, AZ (US); Lindsay Kesler, Phoenix, AZ (US); Thomas J. Johnson, Phoenix, AZ (US); Douglas Harkey, Phoenix, AZ (US)

(21) Appl. No.: 17/156,317

(22) Filed: Jan. 22, 2021

Related U.S. Application Data
(60) Provisional application No. 62/965,504, filed on Jan. 24, 2020.

(51) Int. Cl. A61K 33/00; A61K 9/00; A61P 27/04; A61F 9/00

(52) U.S. Cl. CPC A61K 33/00; A61F 9/0008; A61P 27/04; A61K 9/0048

(57) ABSTRACT

Provided herein are compositions comprising hydrogen gas or a mixture of hydrogen gas and oxygen gas, which are useful in ocular therapies and tissue healing.

### RELATED APPLICATIONS

This application claims priority of U.S. Provisional Patent Application No. 62/965,504, filed Jan. 24, 2020, the entire content of which is incorporated herein by reference.

### TECHNICAL FIELD

The present disclosure generally relates to the treatment of ocular conditions, and more particularly relates to compositions, for example gel-, ointment-, or liquid-based compositions, emulsions, or as a gas or vapor, and delivery systems adapted to topically treat ocular conditions.

### BACKGROUND

The human eye is prone to a variety of diseases, syndromes, and other undesirable conditions. It has been discovered that many of these problems are favorably influenced, without being bound by theory, by the anti-inflammatory and other positive effects of hydrogen gas or a mixture of hydrogen gas and oxygen gas. These may include, but are not limited to, ocular sequela, iritis, bacterial infection, viral infection, scleritis, episcleritis, allergies, dry-eye, uveitis, post-surgical or post-operative inflammation or healing, reaction to antibiotic/antiviral treatment, and contact-lens-related irritation or issues, red eye, and healing, or a combination thereof. The compositions provided herein are also useful for reducing vascular endothelial growth factor (VEGF), the treatment of macular degeneration, diabetic retinopathy, or other ocular conditions or diseases, or a combination thereof.

Currently known methods for treating and mitigating such conditions are unsatisfactory in a number of respects, including toxicity and allergic reactions. For example, there is a long-felt need for topical solutions that are more therapeutically effective, better tolerated, and safer than the traditional aqueous solutions currently available, including those that promote healing. For example, steroids, the current drug class used to reduce inflammation, are widely known to cause cataracts and elevated intraocular pressure. Dry eye prescription medications available on the market are largely ineffective and do little to reduce patient symptoms and clinical signs of the disease. Furthermore, the use of eye-drops and other conventional delivery methods are difficult to use by the patient and often result in incomplete contact of the treatment solution with the eye being treated.

Accordingly, systems and methods are needed that overcome these and other limitations of currently available ocular treatments.

### SUMMARY

Various embodiments of the present disclosure relate to therapeutically effective compositions and delivery methods for treating ocular conditions utilizing a solution of hydrogen knallgass (2:1 HHO ((H₂)₂:O₂)) or molecular hydrogen (H₂), a saline solution (e.g., a phosphate-buffered saline) or distilled H₂O, and an optional third component (such as mineral oil, aloe, propylene glycol, polyethylene glycol, or ophthalmic lubricants). In some embodiments, the compositions are provided as an emulsion. In various embodiments, the compositions provided herein comprise 0.5-10.0 ppm hydrogen (H₂). In some embodiments, the compositions comprise 0.5-1.6 ppm hydrogen gas. In some embodiments, the compositions are delivered using a single-use cup configured to tightly fit over a human eye region and a quick-release seal that allows the HHO or molecular hydrogen included in the composition to immediately bathe a patient's eye. In some embodiments, the presently described compositions may be administered as a drop (i.e. via topical instillation), an ointment, an eye soak, and emulsion, or as a gas or vapor. In some embodiments, the compositions may be combined with one or more antibiotics, one or more steroids, or a combination thereof. The hydrogen in the solution has been found to down-regulate pro-inflammatory cytokines associated with various ocular conditions.

In embodiments, the present disclosure may be used for the reduction of VEGF (vascular endothelial growth factor) as well as the treatment of macular degeneration and diabetic retinopathy.

In some embodiments, the present disclosure may be used for treatment of, or concurrent with treatment of, ocular sequela, iritis, bacterial infection, viral infection, scleritis, epi-scleritis, allergies, dry-eye, uveitis, post-surgical inflammation, reaction to antibiotic/antiviral treatment, and contact-lens-related irritation and red eye, and as a VEGF inhibitor.

In some embodiments, the present disclosure may be used to promote ocular healing needed as a result of physical damage or another ocular treatment, or irritation due to an ocular disease or another ocular treatment.

### BRIEF DESCRIPTION OF THE DRAWINGS

The present disclosure will hereinafter be described in conjunction with the appended figures, wherein like numerals denote like elements, and:

FIG. 1 is schematic overview of an ocular delivery system in accordance with various embodiments.

FIG. 2A shows, in sequence with FIG. 2B, the application of an HHO or molecular hydrogen (H₂) solution using an ocular delivery system in accordance with various embodiments.

FIG. 2B shows removal of the quick-release seal attached to the opening of the container in FIG. 2A.

### DETAILED DESCRIPTION

The following detailed description of the present disclosure is merely exemplary in nature and is not intended to limit the present disclosure or the application and uses of the present disclosure. Furthermore, there is no intention to be bound by any theory presented in the preceding background or the following detailed description.

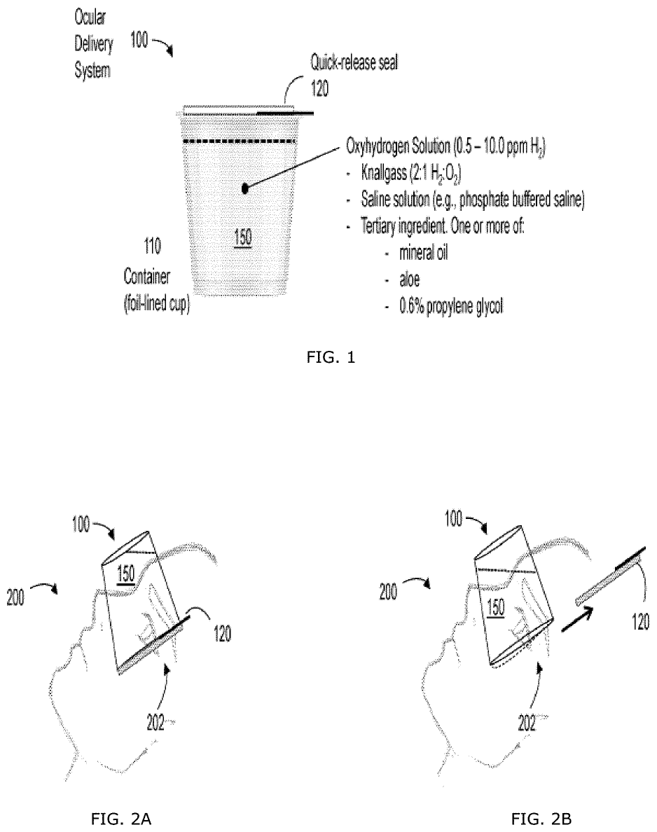

Various embodiments of the present disclosure relate to compositions and delivery methods for treating ocular conditions. Referring first to FIG. 1, an ocular delivery system 100 in accordance with various embodiments generally includes a container 110 (e.g., a foil-lined, one-time-use sterile cup) having a quick-release seal 120 covering its top opening, wherein the geometry of the top opening (e.g., size and shape) is selected to substantially fit within the orbit over a human eye in a reasonably water-tight manner. While FIG. 1 illustrates a traditional cup-shaped container 110, the present disclosure is not so limited. Container 110 may be elliptical, curvilinear, or any other shape suitable for fitting over a human eye.

Quick-release seal 120 may include any combination of components that allow a patient or caregiver to quickly and easily release the eye-treatment solution when container 110 is in contact with the patient's face so that the solution can effectively flow over and bathe the patient's eye. Various adhesives and seal materials may be used.

Disposed within container 110 is an eye-treatment solution (e.g., an HHO or molecular hydrogen (H₂) solution) 150 comprising, for example, hydrogen knallgass or H₂, a saline solution, and one or more optional components such as mineral oil, aloe, propylene glycol, polyethylene glycol, castor oil, or other ophthalmically acceptable lubricants. In this regard, the phrase “oxyhydrogen” as used herein refers to any gaseous combination of diatomic hydrogen and oxygen (H₂, O₂), and the phrase “oxyhydrogen solution” refers to any combination of those gases with a liquid constituent, regardless of whether the resulting mixture is properly classified as a suspension, a solution, a colloid, or any other such mixture of gaseous and liquid components. Furthermore, the term “hydrogen knallgass” (sometimes referred to informally as “HHO”) is used in the sense of an oxyhydrogen gas that has a stoichiometric H₂:O₂ ratio substantially equal to 2:1, which may be produced, for example, through dissociation of water molecules via water electrolysis:
2 H₂O→2 H₂+O₂

HHO is, by definition, the gaseous stoichiometric mixture of hydrogen, hydrogen and oxygen as the result of the disassociation of the covalent bonds of a water molecule through electrolysis or resonant frequency with high voltage and low current.

Thus, in some embodiments, provided herein are compositions comprising: a hydrogen gas; and a vehicle. The compositions may be sterile.

In some embodiments, the hydrogen gas is a combination of hydrogen gas and oxygen gas (O₂). When hydrogen and oxygen gases are both present, the ratio of the gases may be about 2:1 H₂:O₂.

In some embodiments, the hydrogen gas is present at an amount of about 0.5-10 ppm. In some embodiments, the hydrogen gas is present at an amount of about 0.5-2.0 ppm. In some embodiments, the hydrogen gas is present at an amount of about 0.7-1.6 ppm. In some embodiments, the hydrogen gas is present, but in an amount not more than 2.0 ppm, i.e. less than 2.0 ppm. In some embodiments, the hydrogen gas is present at an amount of about 1.6 ppm.

In some embodiments, the hydrogen gas is present at an amount of about 2 ppm, about 3 ppm, about 4 ppm, about 5 ppm, about 5-10 ppm, or about 10 ppm. In some embodiments, the hydrogen gas is present at an amount of about 20 ppm.

In some embodiments, the vehicle is water. In some embodiments, the water is distilled water, deionized water, or both. In some embodiments, the vehicle is a phosphate buffered saline, which may include calcium, magnesium, or both. In some embodiments, the vehicle is a ringer's solution, which may include magnesium. In some embodiments, the ringer's solution is a lactated ringer's solution. In some embodiments, the vehicle may be sterile.

In some embodiments, the pH of the compositions provided herein may be about 6.0-8.0. In some embodiments, the pH of the compositions is about 6.5-7.5. In some embodiments, the pH of the compositions is about 6.5. In some embodiments, the pH of the compositions is about 7.2. In some embodiments, the pH of the compositions is about 7.3-7.4.

In some embodiments, the compositions provided herein further include one or more additional components. In some embodiments, the additional component is an ophthalmic lubricant. In some embodiments, the additional component is, independently, mineral oil, aloe, propylene glycol, or polyethylene glycol.

In some embodiments, the composition is a mixture of about 0.7-1.6 ppm H₂ in sterile distilled water having a pH of about 7.2. In some embodiments, the composition is a mixture of about 0.7-1.6 ppm H₂, O₂, and sterile distilled water, wherein the ratio of H₂:O₂ is about 2:1, and the pH of the composition is about 7.2. In some embodiments, these compositions further comprise an aloe, a mineral oil, or both. In some embodiments, these compositions further comprise erythromycin.

In some embodiments, the composition is a mixture of about 5-10 ppm H₂ in sterile distilled water having a pH of about 6.5-7.5. In some embodiments, the composition is a mixture of about 5-10 ppm H_{z}, O₂, and sterile distilled water, wherein the ratio of H₂:O₂ is about 2:1, and the pH of the composition is about 6.5-7.5. In some embodiments, these compositions further comprise an aloe, a mineral oil, or both. In some embodiments, these compositions further comprise erythromycin.

Distilled water may have an oxygen gas concentration of 6.5-8 mg/L. Thus, in some embodiments, the composition comprises an oxygen gas concentration of about 9-13 mg/L. In some embodiments, the composition comprises an oxygen gas concentration of at least 9 mg/L to about 13 mg/L. Thus, in some embodiments, the ratio of H_{z}:O₂ is about 2:1, and the pH of the composition is about 6.5-7.5, and the concentration of O₂ is about 9-13 mg/L. In some embodiments, the O₂ concentration is about 9 mg/L. In some embodiments, the O₂ concentration is about 10 mg/L. In some embodiments, the O₂ concentration is about 11 mg/L. In some embodiments, the O₂ concentration is about 12 mg/L. In some embodiments, the O₂ concentration is about 3 mg/L.

In various embodiments, eye-treatment solution comprises, overall, 0.5-10.0 ppm H_{z}, and the saline solution consists of medical grade phosphate-buffered saline as is known in the art. In addition to hydrogen knallgass and a saline solution, eye-treatment solution 150 may also include various other ingredients (“tertiary ingredients”), such as mineral oil, aloe, and propylene glycol, each in various weight or volume-percents ranging from about 5% to 7%, though other embodiments may incorporate other ingredients at different weight or volume percents.

In accordance with a first embodiment, eye-treatment solution 150 comprises hydrogen or knallgass, and phosphate buffered saline combined such that the hydrogen concentration is in the range of 0.5-10.0 ppm.

In accordance with a second embodiment, eye-treatment solution 150 comprises hydrogen or knallgass, phosphate buffered saline, and mineral oil (e.g., 1% by weight) combined such that the hydrogen concentration is in the range of 0.5-10.0 ppm.

In accordance with a third embodiment, eye-treatment solution 150 comprises hydrogen or knallgass, phosphate buffered saline, and an aloe (e.g., aloe vera liquid) combined such that the hydrogen concentration is in the range of 0.5-10.0 ppm.

In accordance with a fourth embodiment, eye-treatment solution 150 comprises hydrogen or knallgass, phosphate buffered saline, and 0.6% propylene glycol combined such that the hydrogen concentration is in the range of 0.5-10.0 ppm.

In accordance with a fifth embodiment, eye-treatment solution 150 comprises hydrogen or knallgass, phosphate buffered saline, aloe, and 0.6% propylene glycol combined such that the hydrogen concentration is in the range of 0.5-10.0 ppm.

Table 1 provides examples of selected compositions provided herein.

TABLE 1
Compositions

| | Gas (H₂ 0.7-1.6 | | |
|---|---|---|---|
| | ppm; H₂:O₂ of | | |
| | about 2:1 when | | Additional |
| Composition | present) | Vehicle | components |
| 1 | H₂ | Sterile distilled | |
| | | water; pH about | |
| | | 7.2 | |
| 2 | H₂ | Phosphate buffered | |
| | | saline (PBS) (137 | |
| | | mM NaCl, 10 mM | |
| | | phosphate, 2.7 mM | |
| | | KCl; pH 7.4) | |
| 3 | H₂ | Ringer's solution | |
| 4 | H₂ | Sterile distilled | Mineral oil |
| | | water | |
| 5 | H₂ | PBS | Mineral oil |
| 6 | H₂ | Ringer's solution | Mineral oil |
| 7 | H₂ | PBS | Mineral oil |
| | | | Aloe |
| | | | Propylene glycol |
| 8 | H₂ and O₂ | Sterile distilled | |
| | | water; pH about | |
| | | 7.2 | |
| 9 | H₂ and O₂ | PBS | |
| 10 | H₂ and O₂ | Ringer's solution | |
| 11 | H₂ and O₂ | Sterile distilled | Mineral oil |
| | | water | |
| 12 | H₂ and O₂ | PBS | Mineral oil |
| 13 | H₂ and O₂ | Ringer's solution | Mineral oil |
| 14 | H₂ and O₂ | PBS | Mineral oil |
| | | | Aloe |
| | | | Propylene glycol |

Treatment of a patient for an ocular condition generally proceeds as illustrated in FIGS. 2A and 2B. The ocular delivery system 100 is first positioned with the quick-release seal 120 facing downward and the perimeter of the opening of container 110 in contact with the patient's face 200 in and around eye area 202 (FIG. 2A). Subsequently, as shown in FIG. 2B, the patient (or caregiver) actuates quick-release seal 120 such that the eye-treatment solution 150 is allowed to freely flow, under the force of gravity, into and around the eye region 202. While some leakage of solution 150 is expected, the seal provided between the opening of container 110 and the patient's face 200 substantially restrains the solution 150 so that it can fully cover the eye region for a significant length of time, unlike eye drops that contact the eye only briefly.

Due to the improved efficacy of this delivery system, combined with the therapeutic level of hydrogen provided by eye-treatment solution 150, the result is an ocular treatment system the functions to down-regulate pro-inflammatory cytokines associated with various ocular conditions, such as allergies, dry-eye, epi-scleritis, scleritis, uveitis, post-surgical inflammation, antibiotic treatments, and contact-lens-related irritation (e.g., red-eye).

Alternatively, use of the compositions provided herein (i.e. compositions of Table 1) for treating or healing as described herein may be by topical administration as a drop (i.e. ocular instillation), an ointment, or as a soak. In some embodiments, the compositions are used on an eye of a subject. The subject may be a human, or another animal. In some embodiments, the animal is a domesticated animal, including a livestock, or a pet.

### EXAMPLES

### Example 1: Assessment of a Hydrogen/Oxygen Solution and a Hydrogen Solution on Irritated Rabbit Corneas

Topical 0.1% benzalkonium chloride (BAC) are administered to the eyes of male New Zealand white rabbits as a model for induced ocular irritation, as described in Table 2. In ophthalmology, stains are applied to the surface of the eye and examined with a microscope to evaluate and quantify the extent of dryness or other ocular pathology. The industry standard for assessing the severity of dry eye syndrome is lissamine green stain. Fluorescein stain is also widely used.

Table 2 shows the study design. Four rabbits are in each of the three groups; each groups dosing route being topical ocular. Group 1 is the control group with BAC challenge, and sterile water soak. Group 2 receives the BAC challenge, and HHO (composition 8) soak. Group 3 receives the BAC challenge, and H2 (composition 1) soak.

A total of 20 animals are administered BAC OD on Days 1 through 24 of the study; administered three times daily, approximately 4 hours apart, for 14 days (days 1 through 14), followed by once daily for 10 days. Following the Day 24 ophthalmic exam, 12 animals were selected for study based upon conditioning, ophthalmic findings, and staining scores. All doses were administered at approximately the same time each day ±5 minutes. Legend: BAC (benzalkonium chloride); OD (right eye); OU (both eyes); SDW (sterile distilled water); HHO (hydrogen and oxygen solution; composition 8); H2, Hydrogen solution (composition 1); NA (not applicable); (a) administered three times daily, approximately 4 hours apart, for 14 days (days 1 through 14), followed by once daily for 24 days (days 16 through 37 in the a.m.), as a 0.1% solution to generate a dry eye condition; (b) administered once daily beginning on day 25 for 13 days (days 25 through 37)—the dose was in the P.M. after the BAC dose for days 25 through 37; and (c) administered four times daily, approximately 2 hours apart, beginning on day 25 for 13 days (days 25 through 37)—the HHO (group 2) or H2 (group 3) doses were after the BAC dose for days 25 through 37.

TABLE 2
Study design

| | | | Target Dose | Target Dose | |
|---|---|---|---|---|---|
| | | | Level | Volume | Sample |
| Group | Days | Treatment | (mg/eye/dose) | (μL/eye) | Collections |
| 1 | 1-37 | BAC OD^{a} | 0.02 | 20 | Tears, |
| | 25-37 | SDW OU^{b} | NA | 500 | Eyes |
| 2 | 1-37 | BAC OD^{a} | 0.02 | 20 | Tears, |
| | 25-37 | HHO OU^{c} | NA | 500 | Eyes |
| 3 | 1-37 | BAC OD^{a} | 0.02 | 20 | Tears, |
| | 25-37 | H2 OU^{c} | NA | 500 | Eyes |

Topical administration of 0.1% BAC to the right eyes results in a notable and prolonged increase in corneal fluorescein and lissamine green staining scores. In the left eyes, which are not administered BAC, corneal fluorescein and lissamine green staining scores remain relatively consistent between days 2 and 36, with the exception of day 21, when scores are higher for an undeterminable reason. This stability of staining scores during administration of the vehicle and both test articles indicates that topical administration of sterile distilled water, HHO, or H2 is well tolerated.

For the right eyes of Group 1 animals administered 0.1% BAC and SDW (starting on day 25), corneal fluorescein and lissamine green staining scores remain relatively consistent between days 6 and 36, which indicate that the model system is stable during the dosing phase (days 25 through 36), and no reduction in staining scores attributable to SDW occurred. Topical application of HHO and H2 resulted in a mean reduction in corneal fluorescein and lissamine green staining scores between days 28 and 36 in the right eyes treated with BAC. Combining data over the dosing phase and comparing the groups to each other during the dosing phase better controls for some of the day to day variability in staining scores. When the staining scores for the right eyes from each group are averaged over the entirety of the dosing phase examinations (days 28 through 36), mean corneal fluorescein and lissamine green staining scores for Group 1 administered SDW are 3.83 and 2.17, respectively. These are compared to scores of 3.00 and 1.58 for fluorescein and lissamine green staining, respectively, for the eyes administered HHO and 2.08 and 0.83 for eyes administered H2. These differences in overall mean scores suggest a clear treatment and efficacy effect for H2 and a treatment and efficacy effect for HHO. While eyes treated with HHO and H2 have mean lissamine and fluorescein staining scores that are much better than the control eyes, staining from day 36, the last day of the study, are indisputable. Cumulative staining scores of eyes in the control group stained with lissamine green is 10. Cumulative staining scores of all eye treated with H2 is 1. In other words, eyes treated with H2 have 10× less staining than the control eyes. Results are shown in Tables 3-6.

TABLE 3
Mean Scores for Corneal Fluorescein Staining-Left Eye (Control);
Dosing of SDW, HHO, and H2 Started on Day 25 (N = 4 eyes)

| | Day of Ophthalmic Examination | | | | | | |
|---|---|---|---|---|---|---|---|
| | 0 | 2 | 6 | 8 | 10 | 13 | 15 |
| Group 1 SDW | 0.25 | 1.25 | 1.5 | 3 | 1.25 | 1.25 | 1 |
| Group 2 HHO | 0.25 | 1.5 | 1.5 | 2 | 0.5 | 1.25 | 1.25 |
| Group 3 H2 | 0.5 | 1.25 | 0.75 | 1 | 1 | 1 | 0.75 |

| | Day of Ophthalmic Examination | | | | | |
|---|---|---|---|---|---|---|
| | 17 | 21 | 24 | 28 | 31 | 36 |
| Group 1 SDW | 0.75 | 2.75 | 0.5 | 1.25 | 1 | 1.25 |
| Group 2 HHO | 0 | 2.25 | 0 | 0.25 | 1 | 1.25 |
| Group 3 H2 | 0.5 | 1.75 | 0.25 | 0 | 0.75 | 0.25 |

TABLE 4
Mean Scores for Corneal Fluorescein Staining-Right Eye;
BAC Administration Started After Day 0 Examination, and
Dosing of SDW, HHO, and H2 Started on Day 25 (N = 4 eyes)

| | Day of Ophthalmic Examination | | | | | | |
|---|---|---|---|---|---|---|---|
| | 0 | 2 | 6 | 8 | 10 | 13 | 15 |
| Group 1 BAC + SDW | 0.25 | 2.75 | 3.5 | 4.25 | 4.25 | 5 | 4.5 |
| Group 2 BAC + HHO | 0.25 | 2 | 2.75 | 3.25 | 3.5 | 3.75 | 3.75 |
| Group 3 BAC + H2 | 0.25 | 2.25 | 2.25 | 2.5 | 3.75 | 3.5 | 3.25 |

| | Day of Ophthalmic Examination | | | | | |
|---|---|---|---|---|---|---|
| | 17 | 21 | 24 | 28 | 31 | 36 |
| Group 1 BAC + SDW | 3.25 | 4 | 2.75 | 3.75 | 3.75 | 4 |
| Group 2 BAC + HHO | 3.5 | 3.25 | 2.5 | 3.25 | 3 | 2.75 |
| Group 3 BAC + H2 | 4 | 3.5 | 2.5 | 1.75 | 2.25 | 2 |

TABLE 5
Mean Scores for Lissamine Green Staining-Left Eye; Dosing
of SDW, HHO, and H2 Started on Day 25 (N = 4 eyes)

| | Day of Ophthalmic Examination | | | | | | |
|---|---|---|---|---|---|---|---|
| | 0 | 2 | 6 | 8 | 10 | 13 | 15 |
| Group 1 SDW | 0 | 0 | 0.25 | 0.5 | 0.5 | 0.5 | 0.25 |
| Group 2 HHO | 0 | 0 | 0 | 0.75 | 0 | 0 | 0.5 |
| Group 3 H2 | 0 | 0 | 0.5 | 0.25 | 0 | 0 | 0 |

| | Day of Ophthalmic Examination | | | | | |
|---|---|---|---|---|---|---|
| | 17 | 21 | 24 | 28 | 31 | 36 |
| Group 1 SDW | 0.25 | 1.75 | 0.75 | 0.5 | 0.25 | 0.25 |
| Group 2 HHO | 0 | 1 | 0 | 0.5 | 0.25 | 0.5 |
| Group 3 H2 | 0 | 1.25 | 0.25 | 0 | 0.25 | 0 |

TABLE 6
Mean Scores for Corneal Lissamine Green Staining-Right Eye;
BAC Administration Started After Day 0 Examination, and
Dosing of SDW, HHO, and H2 Started on Day 25 (N = 4 eyes)

| | Day of Ophthalmic Examination | | | | | | |
|---|---|---|---|---|---|---|---|
| | 0 | 2 | 6 | 8 | 10 | 13 | 15 |
| Group 1 BAC + SDW | 0 | 0.25 | 2.75 | 2.25 | 2.25 | 3.25 | 2 |
| Group 2 BAC + HHO | 0 | 0 | 1.5 | 2 | 1 | 1.5 | 1.75 |
| Group 3 BAC + H2 | 0 | 0 | 1 | 0.5 | 0.75 | 0.5 | 2.25 |

| | Day of Ophthalmic Examination | | | | | |
|---|---|---|---|---|---|---|
| | 17 | 21 | 24 | 28 | 31 | 36 |
| Group 1 BAC + SDW | 1.25 | 1.75 | 1.75 | 2 | 2.25 | 2.5 |
| Group 2 BAC + HHO | 1.5 | 2.0 | 1.0 | 1.75 | 1.5 | 1.5 |
| Group 3 BAC + H2 | 1.5 | 2.25 | 1.0 | 0.5 | 1.25 | 0.25 |

While the illustrated embodiments are described in the context of a human eye, the present disclosure is not so limited. The delivery system, including the geometry of the container, quick-release seal, and the like may be adapted for the treatment of a variety of animals prone to eye conditions, such as dogs, cats, and other mammals.

The foregoing detailed description will provide those skilled in the art with a convenient road map for implementing various embodiments of the present disclosure. However, it will be appreciated that the particular embodiments described above are only examples, and are not intended to limit the scope, applicability, or configuration of the present disclosure in any way. To the contrary, various changes may be made in the function and arrangement of elements described without departing from the scope of the present disclosure.

As used herein, the term “about” in reference to a numerical value means±10% of the given numerical value. Thus, about 1.0 refers to a range of 0.9-1.1, and about 10 refers to a range of 9-11, for example.

As used herein, the word “exemplary” means “serving as an example, instance, or illustration.” Any implementation described herein as “exemplary” is not necessarily to be construed as preferred or advantageous over other implementations, nor is it intended to be construed as a model that must be literally duplicated.

The term “treatment” refers to the application of one or more specific procedures used for the amelioration of a disease. In certain embodiments, the specific procedure is the administration of one or more therapeutic agents. “Treatment” of a subject (e.g. a mammal, such as a human) or a cell is any type of intervention used in an attempt to alter the natural course of the individual or cell. Treatment includes, but is not limited to, administration of a composition (e.g. a pharmaceutical composition), and may be performed either prophylactically or subsequent to the initiation of a pathologic event or contact with an etiologic agent. Treatment includes any desirable effect on the symptoms or pathology of a disease or condition, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset. An “effective amount” or “therapeutically effective amount” refers to an amount of therapeutic compound, such as hydrogen gas or hydrogen gas and oxygen gas, administered to a subject, either as a single dose or as part of a series of doses, which is effective to produce a desired therapeutic effect.

We claim:

1. A composition, comprising:
a therapeutically effective amount of hydrogen gas; and
a saline solution or sterile water,
wherein the composition is suitable for topical application to a human eye.

2. The composition of claim 1, further comprising oxygen gas.

3. The composition of claim 1, wherein the hydrogen gas and oxygen gas have an H₂:O₂ ratio of about 2:1.

4. The composition of claim 1, wherein the composition includes from 0.5 to 10.0 ppm H₂.

5. The composition of claim 1, wherein the composition includes about 5-10 ppm H₂.

6. The composition of claim 1, wherein the composition includes about 0.7-1.6 ppm H₂.

7. The composition of claim 1, further including at least one tertiary ingredient selected from the group consisting of mineral oil, aloe, polyethylene glycol, and propylene glycol.

8. The composition of claim 1, wherein the saline solution comprises phosphate buffered saline.

9. The composition of claim 1, wherein the saline solution comprises a ringer's solution.

10. The composition of claim 1, wherein the pH of the composition is about 6.5.

11. The composition of claim 1, wherein the pH of the composition is about 7.2-7.4.

12. The composition of claim 1, wherein the composition comprises an oxygen gas concentration of about 9-13 mg/L.

13. The composition of claim 1, wherein the composition comprises an oxygen gas concentration of about 9-13 mg/L, and the pH of the composition is about 7.2-7.4.

14. An ocular delivery system comprising:
a container having an opening configured to substantially fit over an eye of a subject;
a quick-release seal removably attached to, and providing a water-proof seal for, the opening of the container; and
a composition disposed within the container.

15. An ocular delivery system comprising:
a container having an opening configured to substantially fit over an eye of a subject;
a quick-release seal removably attached to, and providing a water-proof seal for, the opening of the container; and
the composition of claim 1 disposed within the container.

16. A method of treating an ocular condition, the method comprising:
providing the ocular delivery system of claim 15;
placing the ocular delivery system over the eye of the subject; and
deploying the quick-release seal to expose the eye of the subject to the composition.

17. A method of healing or promoting healing of an ocular surface, the method comprising:
providing the ocular delivery system of claim 15;
placing the ocular delivery system over the eye of the subject; and
deploying the quick-release seal to expose the eye of the subject to the composition.

18. A method of treating an ocular disease in a subject in need thereof, comprising administering an effective amount of the composition of claim 1 to an eye of the subject.

19. A method of reducing vascular endothelial growth factor (VEGF) in a cell, comprising contacting the cell with an effective amount of the composition of claim 1.
